(19) United States
(12) Patent Application Publication
(10) Pub. No.: US 2009/0087461 A1
(43) Pub. Date: Apr. 2, 2009

(54) ANTI-BACTERIAL PYROCATECHOLS AND RELATED METHODS

(76) Inventors: Thomas James Boyd, Metuchen, NJ (US); Guofeng Xu, Plainsboro, NJ (US); Ravi Subramanyam, Belle Mead, NJ (US); Joe Vazquez, Hamllton, NJ (US)

Correspondence Address: COLGATE-PALMOLIVE COMPANY, 909 RIVER ROAD, PISCATAWAY, NJ, 08855, US

(21) Appl. No.: 11/865,202

(22) Filed: Oct. 1, 2007

(51) Int. Cl. A61K 8/02; C07C 39/04; A61K 31/05; A61Q 11/00; A61K 8/34

(52) U.S. Cl. 424/401; 568/716; 424/49; 514/731

(57) ABSTRACT

The invention includes a compound or an oral care composition comprising a compound represented by the structure (I):

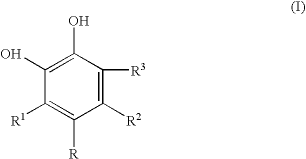

wherein R³ is selected from a hydrogen atom and a structure represented by:

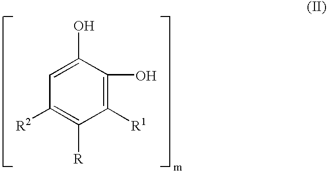

wherein m is an integer of 0 to 100, R is independently selected from a first hydrocarbon structure having from 1 to 50 carbon atom and R1 and R2 are independently chosen from a hydrogen atom and a second hydrocarbon structure having 1 to 10 carbon atoms. Related methods are also disclosed.

### BACKGROUND OF THE INVENTION

Effective and safe anti-bacterial agents are important to the personal care industry, especially for oral care. A number of disease conditions are associated with the action of bacteria in the oral cavity. Dental plaque, gingivitis, periodontitis, and tartar are several known conditions associated with bacteria in the oral cavity.

To prevent or treat these disease conditions, anti-bacterial agents ale often incorporated into oral care compositions. Often these anti-bacterial agents are reported as having a lack of activity, e.g., not providing a robust reduction of bacteria or bacterial by-products, including volatile sulfur compounds (“VSC”). In some cases, otherwise effective agents cannot be formulated due to factors such as limited solubility and hence bioavailability, cationic charge (which limits use in oral care products), and poor safety profile.

### BRIEF SUMMARY OF THE INVENTION

The invention includes a compound or an oral care composition comprising a compound represented by the structure (I):

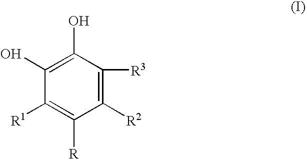

wherein R³ is a hydrogen atom or is represented the by structure (II):

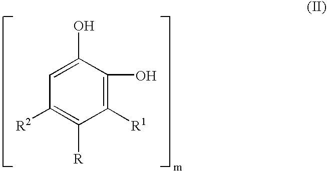

wherein m is an integer of 0 to 100, R is independently selected from a first hydrocarbon structure having from 1 to 50 carbon atom and R¹ and R² are independently chosen from a hydrogen atom and a second hydrocarbon structure having 1 to 10 carbon atoms.

Also included are methods of using the compound of structure (I), including a method of reducing a bacterial population on a substrate comprising contacting the substrate with a compound represented by the structure (I).

Alternatively, the invention includes a method of maintaining and/or facilitating the systemic health of a mammal comprising contacting a surface of the oral cavity of the mammal with a composition that comprises a compound represented by the structure (I).

### DETAILED DESCRIPTION OF THE INVENTION

As used throughout, ranges are used as a shorthand for describing each and every value that is within the range. Any value within the range can be selected as the terminus of the range.

The invention includes the compound (I) which is represented by the structure:

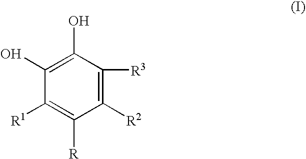

wherein R³ is represented by the structure (II):

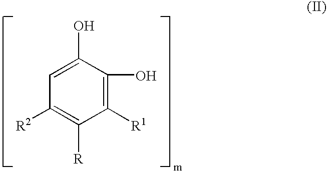

The symbol “m” in structure (I) represents an integer of 0 to 100, of 1 to 20, of 1 to 15, or of 1, 2, 3, 4, 5, 6, 7, 8, 9, or 10. R may independently represent any hydrocarbon structure known or to be developed in the art. It may be preferred that the hydrocarbon structure of R has 1 to 50 carbon atoms, 1 to 20 carbons, or 1 to 10 carbon atoms. Additionally, if desired, R may be an alkyl group, an alkoxy group, an alkene group, an alkyne group, and/or a alkane group.

The compound (I) includes R¹ and R², which represent independently a hydrogen atom or a second hydrocarbon structure having 1 to 10 carbon atoms. R¹ and R² may be the same in each monomer of the compound (I), or they may be different. R¹ and R² may be independently chosen from an alkyl group, an alkoxy group, an alkene group, an alkyne group, and/or a alkane group.

The hydrocarbon structures of any of R, R¹, R², and R³ may be independently ring structures, chain structures, linear structures, branched structures or combinations of these. Any of the carbon atoms within the hydrocarbon structures of R, R¹, R², and R³ and/or the entire compound (I) may be independently substituted or unsubstituted with any functional group(s) known in the art. Methyl, ethyl, butyl, hydroxy, alkyl and halogen groups may be preferred functional groups.

The invention may include a compound of the structure (III):

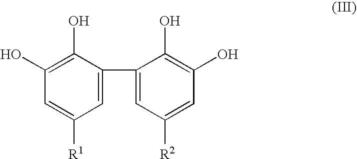

where R¹ and R² are independently chosen from a hydrogen atom, an alkenyl group and an alkyl group or of the structure (IV):

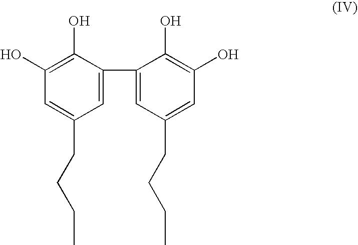

The compounds described above may be synthesized by any suitable pathway or synthesis process or may be isolated or purified from a natural source. For example, the compound of the invention may be prepared by a simple Friedel-Crafts type acylation of the parent pyrocatechol, followed by reduction to yield the desired end product.

The invention includes an oral care composition containing at least one of the compounds represented by formulae above and a suitable carrier. Such carrier may include all of the components of the oral composition except for the active agent, such as, for example, inactive ingredients, vehicles. The carrier may be or may include, water, glycerin, salts, polyethylene glycol, fumed silica, polymers, marine colloids, gums acrylate polymers, cellulose polymers, starches, gelatins, oils, surfactants, materials that dissolve upon exposure to the oral environment, a textile substrate, and a fiber and other excipients. The carrier may be in the form of a gel, a liquid, a paste, a bead, a lozenge, a chewing gum, a chewable confectionary (“chewie”), a foam, and a spray (aerosolized or non-aerosolized) and a solid.

The compound (I) of the invention may be present in any amount in the oral care composition. It may be desirable that it is included in an amount of about 0.001 wt. % to about 10 wt. %, based on the total weight of the oral care composition, for example from 0.01 wt. % to about 5 wt. % or about 0.1 wt. % to about 2 wt. %. The effective amount may vary depending on the form of the oral composition. For example, in toothpastes, tooth gels, mouth rinses, lozenges and tooth powders, an effective amount may be at least about 0.01 wt. % and or at least about 0.05 wt. %.

If desired, the compound or the oral composition of the invention may be coated onto to and/or impregnated within an oral care implement, such as a fiber or floss, a bristle, tongue and/or soft tissue cleaning elements, mouthguards, and/or orthodontic or prosthetic implants or elements.

In addition to the antibacterial compound, a number of active ingredients and functional materials may be included in the oral care compositions. Such materials include, without limitation, abrasives, humectants, surfactants, anticalculus agents, thickeners, viscosity modifiers, anticaries agents, flavorants, colorants, additional antibacterial agents, antioxidants, anti-inflammation components, and so on. Such materials may be added to the pastes, rinses, gums, lozenges, strips, and other forms of the oral care compositions according to known methods.

According to some embodiments, where the carrier of the oral care composition is solid or a paste, the oral composition includes a dentally acceptable abrasive material, which serves to either polish the tooth enamel or provide a whitening effect. Non-limiting examples include silica abrasives such as silica gels and precipitated silicas. Commercial embodiments include ZEODENT®115, marketed by J. M. Huber, Edison, N.J., United States of America, and SYLODENT®XWA, SYLODENT® 783 or SYLODENT® 650 XWA of the Davison Chemical Division of W.R. Grace & Co., New York, N.Y., United States of America. Other suitable dentifrice abrasives include, without limitation, sodium metaphosphate, potassium metaphosphate, tricalcium phosphate, dihydrated dicalcium phosphate, aluminum silicate, calcined alumina, bentonite or other siliceous materials, or combinations thereof.

According to some embodiments an oral care composition includes at least one humectant, useful for example to prevent hardening of a toothpaste upon exposure to air. Any orally acceptable humectant can be used, including without limitation polyhydric alcohols such as glycerin, sorbitol, xylitol and low molecular weight PEGs. In some embodiments, sne or more humectants are present in a total amount of about 1 wt. % to about 70 wt. %, for example about 1 wt. % to about 50 wvt.%, about 2 wt. % to about 25 wt. %, or about 5 wt. % to about 15 wt. %.

An oral care composition may also include at least one surfactant. In some embodiments, a surfactant may provide enhanced stabilith, help in cleaning the dental surface through detergency, and provide foam upon agitation, e.g., during brushing with a dentifrice composition of the invention. Any orally acceptable surfactant, most of which are anionic, nonionic or amphoteric, can be used. Suitable anionic surfactants include without limitation water-soluble salts of C₈₋₂₀ alkyl sulfates, sulfonated monoglycerides of C₈₋₂₀ fatty acids, sarcosinates, taurates, and the like. Illustrative examples of these and other classes may include sodium lauryl sulfate, sodium coconut monoglyceride sulfonate, sodium lauryl sarcosinate, sodium lauryl isethionate, sodium laureth carboxylate and sodium dodecyl benzenesulfonate. Suitable nonionic surfactants may include without limitation poloxamers, polyoxyethylene sorbitan esters, fatty alcohol ethoxylates, alkylphenol ethoxylates, tertiary amine oxides, tertiary phosphine oxides, dialkyl sulfoxides and the like. Suitable amphoteric surfactants may include without limitation derivatives of C₈₋₂₀ aliphatic secondary and tertiary amines having an anionic group such as carboxylate, sulfate, sulfonate, phosphate or phosphonate. A suitable example is cocoamidopropyl betaine. In some embodiments, one or more surfactants are present in a total amount of about 0.01 wt. % to about 10 wt. %; for example about 0.05 wt. % to about 5 wt. %; or about 0.1 wt. % to about 2 wt. %.

In another embodiment, the composition includes an orally acceptable anticalculus agent. One or more such agents may be present in various embodiments. Suitable anticalculus agents may include without limitation phosphates and polyphosphates (for example pyrophosphates), polyaminopropanesulfonic acid (AMPS), zinc citrate trihydrate, polypeptides such as polyaspartic and polyglutamic acids, polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example the alkali metal and ammonium salts. Suitable inorganic phosphate and polyphosphate salts may include, for example, monobasic, dibasic and tribasic sodium phosphates, sodium tripolyphosphate (STPP), tetrapolyphosphate, mono-, di-, tri- and tetrasodium pyrophosphates, disodium dihydrogen pyrophosphate, sodium trimetaphosphate, sodium hexametaphosphate and the like, wherein sodium can optionally be replaced by potassium or ammonium in some embodiments.

The oral care composition of the invention may include polycarboxylate polymers. Polycarboxylate polymers may include polymers or copolymers of monomers that contain carboxylic acid groups, such as acrylic acid, methacrylic acid, and maleic acid or anhydride. Non-limiting examples may include polyvinyl methyl ether/maleic anhydride (PVME/MA) copolymers, such as those available under the GANTREZ® brand from ISP, Wayne, N.J., United States of America. Still other useful anticalculus agents may include sequestering agents including hydroxycarboxylic acids such as citric, fumaric, malic, glutaric and oxalic acids and salts thereof, and aminopolycarboxylic acids such as ethylenediaminetetraacetic acid (EDTA).

In some embodiments, a composition of the invention includes at least one thickening agent. In some embodiments, a thickener may impart a desired consistency and/or mouth feel to the oral care composition. Any orally acceptable thickening agent may be used, including without limitation carbomers, also known as carboxyvinyl polymers, carrageenans, cellulosic polymers such as hydroxyethylcellulose, carboxymethylcellulose (CMC) and salts thereof, e.g., CMC sodium, natural gums such as karaya, xanthan, gum arabic and tragacanth, colloidal magnesium aluminum silicate, colloidal silica and the like. In some embodiments, one or more thickening agents are present in a total amount of about 0.01 wt. % to about 15 wt. %; for example about 0.1 wt. % to about 10 wt. %; or about 0.2 wt. % to about 5 wt. %.

According to some embodiments, an oral care composition includes at least one viscosity modifier. In some embodiments, a viscosity modifier inhibits settling or separation of ingredients or to promote redispersibility upon agitation of a liquid composition. Any orally acceptable viscosity modifier may be used, including without limitation mineral oil, petrolatum, clays and organo-modified clays, silica, and the like. In some embodiments, one or more viscosity modifiers are present in a total amount of about 0.01 wt. % to about 10 wt. %; for example about 0.1 wt. % to about 5 wt. %.

In another embodiment, the composition includes an orally acceptable source of fluoride ions. In some embodiments, one or more such sources are present. Suitable sources of fluoride ions include fluoride, monofluorophosphate and fluorosilicate salts, and amine fluorides, including olaflur (N′-octadecyltrimethylendiamine-N,N,N′-tris(2-ethanol)-dihydrofluoride). Any such salt that is orally acceptable may be suitable, including without limitation alkali metal (e.g., potassium, sodium), ammonium, stannous and indium salts, and the like. In some embodiments, water-soluble fluoride-releasing salts are used. According to some embodiments, one or more fluoride-releasing salts are present in an amount providing a total of about 100 ppm to about 20,000 ppm; about 200 ppm to about 5,000 ppm; or about 500 ppm to about 2,500 ppm, fluoride ions. In some embodiments, where sodium fluoride is the sole fluoride-releasing salt present, the oral care composition includes about 0.01 wt. % to about 5 wt. %; about 0.05 wt. % to about 1 wt. %; or about 0.1 wt. % to about 0.5 wt. % sodium fluoride.

Other components may include, without limitation, flavorants, colorants, and other active ingredients such as antioxidants and anti-inflammation agents. In some embodiments, such additional components are formulated into oral compositions according to known procedures.

The invention includes methods of reducing, eliminating or preventing the development of a bacterial population on a substrate, including Gram-negative, Gram-positive, and/or mixtures of both. The method includes contacting any of the compounds and/or compositions of the invention with the selected substrate. The duration of the contact may be short (a few seconds to a few hours) or the substrate may be coated with, impregnated with or otherwise affixed with the compound or composition of the invention. The substrate may be any in the art including plastics, polymer resins, films, metals, fibers, textiles, woods, paper, porcelain, or ceramic. The substrate may be all or part of any device, implement, furnishing or instrument upon which one wishes to control a bacterial population, including, for example, clothing, such as diapers, undergarments, shoes, medical devices, surgical implements, medical implants, office supplies, diaper bags, feminine products, toilet parts, dishware, food service implements, trash cans, pipes, doors, telephone receivers, computer keyboards, railings, floors, operating theater surfaces, hard surfaces, pet equipment, such as carriers, toys and litter boxes; bathroom and kitchen surfaces, walls, currency, laboratory equipment, and/or ophthalmic and dental devices, implements, implants, and tools, such as contact lenses, dentures, and eyeglasses; and epidermal and epithelial surfaces. In an oral environment, the substrate may be pellicle, enamel and/or oral epithelium.

The invention also includes methods of maintaining and/or facilitating the systemic health of a mammal. Such methods include contacting a surface of the oral cavity (such as a dentinal, enamel, gingival, epithelial, pellicle surface) with the composition or the compound (I) of the invention.

### EXAMPLE 1

A simple rinse was prepared in which the selected compound (I), where m=0, R is an ally/group and R¹ and R² are hydrogenatoms, is dissolved in ethanol and is then formulated as shown in Table 1 below.

### EXAMPLE 2

A matching placebo to the formula of Example 1 was prepared according to the formula as shown in Table 1 below.

TABLE 1
Example Rinse Formulations

| Ingredient | Example 1 | Example 2 |
|---|---|---|
| Water | 81.7349% | 81.7849% |
| Ethanol | 10 | 10 |
| Glycerin | 8 | 8 |
| PEG-40 sorbitan diisostearate | 0.125 | 0.125 |
| Flavor | 0.080 | 0.080 |
| Sodium saccharin | 0.010 | 0.010 |
| Dye, 1% solution | 0.0001 | 0.0001 |
| Compound (I) | 0.05% | 0.05% |
| Total | 100% | 100% |

The formulas of Examples 1 and 2 are used to perform an in vitro evaluation of the malodor-reducing capacity. The formulas are each subjected to an oral environment containing VSC. A rinse containing a 0.03% TCN was also subjected to an oral environment containing VSC.

The percent reduction in VSC in the oral environment is measured for each formula. In this case, any reduction in malodor would be expected from superior anti-bacterial activity, especially against Gram-negative bacteria, such as F. nucleatum and P. melangenica, which are well-known to produce VSC in the presence of sulfur-containing amino acids. A very strong impact of the level of oral malodor produced is demonstrated by the formula of Example 1.

What is claimed is:

1. A compound represented by the structure (I): 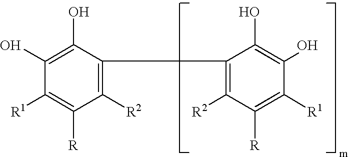
wherein m is an integer of 0 to 100, R is independently selected from a first hydrocarbon structure having from 1 to 50 carbon atom and R¹ and R² are independently chosen from a hydrogen atom and a second hydrocarbon structure having 1 to 10 carbon atoms.

2. An oral care composition comprising a compound represented by the structure (I): 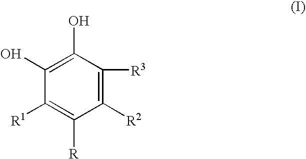
wherein R³ is selected from a hydrogen atom and a structure represented by: 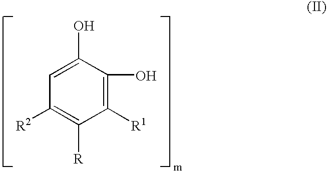
wherein m is an integer of 0 to 100, R is independently selected from a first hydrocarbon structure having from 1 to 50 carbon atom and R¹ and R² are independently chosen from a hydrogen atom and a second hydrocarbon structure having 1 to 10 carbon atoms; and a carrier.

3. The composition of claim 2, wherein m is an integer of 0 to 50.

4. The composition of claim 2, wherein m is an integer of 1 to 20.

5. The composition of claim 2, wherein m is an integer of 1 to 15.

6. The composition of claim 2, wherein m is chosen from 1, 2, 3, 4, 5, and 6.

7. The composition of claim 2, wherein one or both of the first hydrocarbon structure and the second hydrocarbon structure is independently a ring structure.

8. The composition of claim 2, wherein one or both of the first hydrocarbon structure and the second hydrocarbon structure is independently a chain structure.

9. The composition of claim 8, wherein the hydrocarbon chain is independently branched.

10. The composition of claim 2, wherein the first hydrocarbon structure is independently chosen from an alkyl group, an alkoxy group, an alkene group, and alkyne group, and an alkane group.

11. The composition of claim 2, wherein R is independently a hydrocarbon structure having 1 to 20 carbon atoms.

12. The composition of claim 2, wherein R is independently a hydrocarbon structure having 1 to 10 carbon atoms.

13. The composition of claim 2, wherein R¹ and R² are independently chosen from a substituted or unsubstituted methyl group, a substituted or unsubstituted ethyl group, and a substituted or unsubstituted butyl group.

14. The composition of claim 2, wherein the compound (I) is present in the amount of about 0.001% to about 10% by weight.

15. The composition of claim 2, wherein the compound (I) is present in an amount of about 0.01 to about 5% by weight.

16. The composition of claim 2, wherein the compound (I) is present in an amount of about 0.1% to about 2% by weight.

17. The oral care composition of claim 2, further comprising and orally acceptable carrier chosen from a gel, a liquid, a powder, a material that dissolves upon contact with the oral environment, a textile substrate, a fiber, and a paste.

18. The composition of claim 3 further comprising an agent chosen from a co-polymer of polyvinylmethlyether and maleic anhydride, propylene glycol, polyethylene glycol, chitosan, polymer/copolymers of polyvinylphosphonic acid.

19. The oral care composition of claim 2 in the form of a paste, a gel, a lozenge, a liquid, a chewing gum, a chewie, and a spray.

20. A method of reducing a bacterial population on a substrate comprising contacting the substrate with a compound represented by the structure (I): 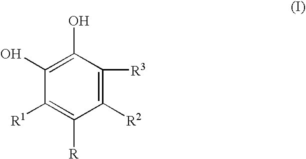
wherein R³ is selected from a hydrogen atom and a structure represented by: 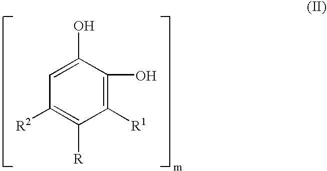
wherein m is an integer of 0 to 100, R is independently selected from a first hydrocarbon structure having from 1 to 50 carbon atom and R¹ and R² are independently chosen from a hydrogen atom and a second hydrocarbon structure having 1 to 10 carbon atoms.

21. The method of claim 19, wherein m is an integer of 1 to 15.

22. The method of claim 19, wherein the first hydrocarbon structure is independently chosen from an alkyl group, an alkoxy group, an alkene group, and alkyne group, and an alkane group.

23. The method of claim 19, wherein the wherein R¹ and R² are independently chosen from a substituted or unsubstituted methyl group, a substituted or unsubstituted ethyl group, and a substituted or unsubstituted butyl group.

24. The method of claim 19, wherein the substrate is chosen from pellicle, enamel and oral epithelium.

25. The method of claim 19, the substrate is a surface of the oral cavity.
